(19) United States
(12) Patent Application Publication
(10) Pub. No.: US 2011/0319878 A1
(43) Pub. Date: Dec. 29, 2011

(54) Red Light Implants for Treating Postpartum Depression

(76) Inventors: Thomas M. DiMauro, Raynham, MA (US); Michael A. Fisher, Grayson, GA (US)

(21) Appl. No.: 12/822,803

(22) Filed: Jun. 24, 2010

(51) Int. Cl. A61B 18/18

(52) U.S. Cl. 606/14

(57) ABSTRACT

Irradiating an orbitofrontal cortex of a patient having postpartum depression with red/near infrared light in order to increase metabolism, bcl-2 and BDNF in the patient's orbitofrontal cortex.

The loving connection between a mother and her baby is a special bonding that can benefit the baby not only in the present, but also well into the future. Bonding brings the mother and child closer together, and this positive attachment can enhance the baby's well being and later development. Because a healthy bond between the mother and her newborn infant is crucial to the proper development of the child, loving efforts to strengthen that bond are highly valued. Some of the ways in which a healthy mother can show love for her child and promote this bonding is by experiencing joy at her child's smile and by providing appropriate attention to her child's needs.

It has been estimated that over 700,000 mothers are afflicted with postpartum depression (PPD) each year in the United States. PPD is considered to be a major depression, and is characterized by standard depressive features. Typical PPD symptoms include non-responsiveness towards the infant's needs and an absence of joy that is normally associated with healthy parent-child interaction and attachment. Because the first months of life are a critical period for an infant's proper cognitive and emotional development, the lack of attachment and attention towards the infant shown by the PPD mother may cause undesired effects in the child's future behaviours.

During pregnancy, the hormonal balance in the healthy expectant mother is such that she experiences extremely high levels of estrogen throughout her body. These levels of estrogen in the expectant mother may be up to 100 times the normal level. After the birth of the child, the estrogen level in the new mother rapidly decreases over the course of a few days and returns to the normal level of estrogen. Estrogen has been found to be critical to many normal neuronal processes, and has been positively associated with serotonin levels in the brain and brain plasticity. Therefore, and without wishing to be tied to a theory, it is believed that PPD may be caused by an extra-sensitive response in a subset of new mothers to the rapid withdrawal of estrogen from the mother's system.

Antidepressants are often one of the first lines of therapy against PPD. Conventional antidepressants such as tricyclics and selective serotonin reuptake inhibitors (SSRIs) are commonly prescribed for PPD. However, there are many problems associated with the use of these conventional antidepressants for PPD. First, these conventional antidepressants typically alleviate the PPD condition in no more than about 80% of the patients taking them. Second, even when successful, these conventional antidepressants typically take up to 8 weeks be effective. Third, the PPD mother can expect to experience the typical side effects associated with tricyclics and SSRIs. Side effects associated with tricyclics use include dry mouth, dry nose, blurred vision, decreased gastro-intestinal motility and secretion, leading to. Side effects associated with SSRI use include insomnia, weight gain and sexual dysfunction.

In addition, the true benefit of antidperessants may be limited in the context of PPD. Logsdon, J. Womens Health (Larchmt), 2009 January-February. 18(1) 85-90 studied the effect of nortriptyline and sertaline (Zoloft^{R}) upon PPD women and found that although the treatment improved the mother's gratification in the maternal role, they did not improve either self-efficacy or maternal-infant interaction.

Lastly, it has been found that virtually all of these conventional antidepressants are found in the mother's milk, and may be transferred to the infant during nursing. There has been little data on the effect of the nursing mother's antidepressant use upon the child's mental development. Rather than demonstrating safety, the literature appears to conclude that the risk to the nursing child posed by the mother's antidepressant use is outweighed by the risks associated with untreated PPD. However, in some cases, the transfer of some particular antidepressants to mother's milk has been so significant that some investigators have concluded that those particular antidepressants should be avoided by nursing mothers.

Without wishing to be tied to a theory, it is now believed that the effectiveness of many conventional antidepressants may lie in their ability to induce neurotrophins such as brain-derived neurotrophic factor (BDNF), in the patient's brain. For example, Duman reports that chronic antidepressant administration increases the expression of BDNF in the hippocampus, as well as frontal cortex and that induction of BDNF in the hippocampus is observed with different classes of antidepressants, but not with nonantidepressant psychotropic drugs Duman, Eur Psychiatry. 2002 July;17 Suppl 3:306-10. However, Duman, Biol. Psychiatry, 2006, 59, 1116-1127, reports that the induction of BDNF is dependent upon chronic chemical antidepressant treatment, consistent with the time course for the therapeutic action of antidepressants.

One of the most positive downstream effects of BDNF induction is termed neuronal sprouting. Sprouting increases the growth of axons or dendrites from a damaged neuron or from an intact neuron that projects to an area denervated by damage to other neurons.

Electroconvulsive therapy (ECT) is undergoing a limited renewal in the United States and has been suggested as a last line treatment for PPD cases in which conventional antidepressants do not remove the PPD. ECT has a high success rate for general major depression and works very quickly. In line with the high effectiveness and rapid response of ECT, it has been reported ECT produces a 4-fold increase in BDNF in the prefrontal cortex of rats and does so with about 2 hours. However, it has also been reported that ECT produces significant short term memory loss and is not believed to be a long lasting therapy, often requiring additional ECT sessions every few months.

US Published Patent Application No. 20050187595 (Streeter) discloses a method for treating or preventing depression. The method, in preferred embodiments, includes therapy apparatus for treating a patient's brain. The method, in a preferred embodiment, includes irradiating at least a portion of a patient's brain with light energy having an efficacious power density and wavelength. The light energy should be sufficient to cause regulation of neurotransmitters in the brain and/or an upregulation of endogenous compounds in the brain, including neurotrophic factors, that result in neural growth, neurogenesis, and/or plasticity of neural function which leads to a diminishment or elimination of depression and its symptoms, and/or delays, reduces, or eliminates the onset of depression or depressive symptoms. However, Streeter discloses that the therapeutic light is to be delivered by directing light through the scalp of the patient.

US Patent Publication No. 2006/0155348 (DeCharms) discusses using red light to treat Parkinson's Disease and other neurological disorders.

### SUMMARY OF THE INVENTION

PPD investigators have recently presented a number of functional MRI studies (fMRI) that examined maternal responses in healthy mothers to their own infants' pictures or sounds (crying or laughter). In general, the investigators found differential activation of a number of regions present in the lower anterior portion of the brain, including ght medial and lateral orbitofrontal cortex (OFC), the temporal pole and the amygdala. See FIGS. 1 and 2. These papers are reported below:

Noriuchi, Biol. Psychitary (2007), performed fMRI on 13 mothers viewing video clips of their own and other infants who demonstrated different attachment behaviours such as smiling at the infant's mother and crying for her. Noriuchi found that the orbitofrontal cortex (OFC) was specifically involved in the recognition of the mother's own infant. Noriuchi further found that the left OFC was associated with the joyful and happy response of the mother to her own infant while the right OFC was associated with the anxiousness of the mother.

Nitschke, Neurolmage 21 (2004) 583-592 reported on fMRIs of healthy mothers viewing photos of their infants and other infants, and reported that the mothers experience bilateral activation of the OFC when viewing their own infants, and that OFC activation correlated positively with pleasant mood ratings. Nitschke concluded that the data “implicate the orbitofrontal cortex in a mother's affective responses to her infant, a form of positive emotion . . . ”

Bartels,Neurolmage 21 (2004) 1155-1166 used fMRI to measure brain activity in mothers while they viewed pictures of their own and acquainted children, and found activity in the OFC.

Lenzi, Cereb. Cortex, 2008 Oct. 10, examined 16 mothers undergoing fMRI while observing and imitating faces of the own and others' children, and found that joy expressions evoked a response mainly in the right limbic and temporal pole areas, while ambiguous expressions elicited a response in the left high order cognitive and moter areas.

Ranote, Neuroreport, 2004 Aug. 6; 15(11) 1825-9, reported the neural correlates of maternal responsiveness shown by healthy mothers viewing alternating video of their own infant, an unknown infant and a neutral video. Ranote reported finding signal change in the bilateral OFC, amygdala and temporal pole, and concluded that the amygdala and temporal pole may be key sites in mediating a mother's response to her infant.

Minagawa-Kawai, Cerebral Cortex, February 2009, 19, 284-292, reported that mothers viewing their own child versus unfamiliar infants elicited increased activations around the anterior part of the OFC.

Leibenluft, Biol. Psychiatry, 2004, 56, 225-232, investigated the neural response of (healthy) mothers viewing their own child versus a familiar child, and found activation in the amygdala, insula, anterior paracingulate cortex, posterior superior temporal sulcus.

Other studies have examined adult responses to stimuli from unfamiliar infants, and found activations in the amygdala and OFC. For example, Kringelbach, PLoS ONE. 2008 Feb. 27;3(2):e1664, investigated whether adults show specific brain responses to unfamiliar infant faces (as compared to adult faces) , and found that highly specific brain activity occurred in the medial OFC, an area implicated in reward behaviour. Seifritz, Biol. Psychiatry, 2003, 54, 1367-75 investigated the neural responses of parents to the laughing and crying of unfamiliar infants, and found amygadalar activation in parents responding to crying.

Thus, overall, this body of this literature points to the proper functioning of the OFC as being crucial in the healthy maternal response.

In addition, two studies have specifically examined the fMRI response of the PPD mother to various stimuli. In a first study, Silverman examined the fMRI response to negative words of mothers suffering from PPD, and reported hypometabolsim in both the posterior medial OFC and the amygdala of the PPD mother. Of note, Silverman's FIG. 1 also reports hypometabolism in a large portion of a lateral region of the OFC as well (“S” in FIG. 3 herein). However, Silverman does not recognize the hypometabolism of the lateral OFC region. In a second study, Fahim, Bipolar Disorders 2007 9, 541-545, examined the fMRI response of a postpartum psychotic (PPP) mother during passive viewing of emotional film excerpts and compared her fMRI response to that of her healthy twin. Fahim found that a small region in the lateral OFC was hypometabolic in the PPP twin. Of note, Fahim reported that this small OFC region was the only region having a different metabolism.

In addition, King, Neurolmage, 30, 2006, 1069-76, investigated the neural correlates of appropriate violent and compassionate behaviours, and reports that the same circuit involving the amygdala and ventromedial prefrontal cortex was activated when participants acted in a context-appropriate manner. King concluded that this circuit was strongly activitated when humans were engaged in “doing the right thing.”

Therefore, when all of these studies are rationalized, it appears that there exists a fairly contiguous region of gray matter running medially and posteriorly from the lateral OFC through the temporal pole and to the amygdala that appears to play an important role mediating healthy responses of a mother towards her child. See FIG. 3. For the purposes of the present invention, this region will be called the Maternal Zone (MZ). See FIG. 4 In short, it appears that the MZ is activated when the healthy mother is engaged in socially desirable activity towards her child. Moreover, the literature (Silverman and Fahim) appears to report that the MZ is somewhat hypometabolic is PPD and PPP mothers.

Moreover, further examination of FIG. 3 reveals that there appears to be a cluster of data five points (Fahim, Bartles, Nitschke, Minagawa-Kawai and Silverman) situated in the anterior portion of the lateral orbitofrontal cortex. This cluster is of note for a number of reasons. First, the multiplicity of researchers who found statistically significant differences in maternal fMRIs at this site over a range of test conditions indicates that this site may be a lynchpin in the maternal emotional framework. Second, this site appears to be the only one in which researchers found not only the expression of joy in mothers viewing their own babies (Nitschke and Minagawa-Kawai) but also an attenuated response to stimuli in PPD mothers (Silverman and Fahim). Lastly, Moses-Kolko, Fertil. Steril., 2008 March 89(3) 685-92 has reported that the left and right lateral OFC were two of the few sites in the PPD brain to display decreased serotonin receptor binding. Of note, the Moses-Kolko paper appears to lend credence to Fahim's hypothesis that the cause of PPD was related to the estrogen-driven decrease in the serotonergic system in the OFC. Therefore, because the site of this cluster appears to evoke baby-related joy in mothers, have attenuated activity in PPD mothers, and have decreased biochemical activity pertinent to emotion in PPD mothers, this site is a prime candidate for the treatments of the present invention.

In addition, Roelofs, SCAN (2009) 4, 50-58 reports that the left lateral OFC (BA47/12) is implicated in the neural control of social emotional behavior and has a general role in overriding dominant-stimulus-response mappings in favor of rule-driven associations. Roelofs also predicts that disturbances such as social avoidance tendencies involved altered responses of the left lateral orbitofrontal cortex. Therefore, it is possible that improving the activation of the left lateral OFC could improve deficits in the mother-child interaction stemming from such a disturbance.

Therefore, in light of the above studies, and without wishing to be tied to a theory, it is believed that the hypometabolism of MZ plays a predominant and direct role in the depressed state of the PPD mother.

It is further believed that the hypometabolism of the MZ can be reversed by low level laser therapy (“LLLT”) treatment of the MZ with red/near infrared light (“red/NIR light”). In particular, it is believed that red/NIR light will beneficially act upon the MZ through the following avenues:

a) increasing the amount of ATP in the MZ;

b) increasing the amount of BDNF in the MZ;

c) increasing the amount of bcl-2 in the MZ, and

d) increasing the amount of sprouting in the MZ

Oron, Photomed Laser Surg. 2007 Jun;25(3):180-2. (2007) reports that in vitro red/NIR light approximately doubles the amount of ATP in neurons. Since metabolic processes of the brain substantially use ATP as their fuel, it is believed that the increase in ATP afforded by LLLT will help normalize the hypometabolism in the MZ experienced by the PPD mother.

Zhang Cell Physiol Biochem. 2008;22(1-4):215-22 reports that LLLT activates PKC in neurons within one hour of the irradiation (Zhang, 2008). Because it is known that PKC increases serotonin release from synapse (Psychopharmacology (Berl). 1989;99(2): 213-8.) and PKC has been implicated in mediating neuronal plasticity, and that increasing the availability of serotonin provides a positive benefit for depressed patients, the activation of PKC should provide a therapeutic benefit to the PPD mother.

As discussed above, it is now believed that the effectiveness of many conventional antidepression treatments may lie in their ability to induce neurotrophins such as brain-derived neurotrophic factor (BDNF), in the patient's brain. It has been shown that LLLT acts upon neurons to increase BDNF 5X in neurons (Byrnes Lasers Surg Med. 2005 August;37(2):161-71.), and (Anders IEEE J. Quantum Electronics, 14/1 January/February 2008, 118-125). The 5X BDNF induction produced by LLLT compares favorably with the increase in BDNF induced by antidepressants, and is approximately the same level of BDNF induction generated by ECT.

bcl-2 is an anti-apoptotic gene that has been implicated in mediating neuronal plasticity. Manji, Psychopharmacol Bull. 2001 Spring;35(2):5-49 and Manji, Am J Psychiatry. 2005 April;162(4):805-7 report that bcl-2 expression correlates with clinical benefit of antidepressants. In this respect, red light has been shown to increase bcl-2 in neurons (Liang, Neuroscience. 2006 May 12;139(2):639-49,) and (Zhang, supra, 2008)

Further without wishing to be tied to a theory, it is further believed that red/NIR light therapy of the MZ will provide a number of advantages to the PPD mother that are not provided by conventional antidepressants.

First, red/NIR light therapy is a completely non-toxic therapy. Thus, it appears that its use poses no known danger to either the mother or child. Therefore, red/NIR light therapy/LLLT can be used by the nursing mother without any apparent risk to the child.

Second, it is believed that red/NIR light therapy will work much more quickly than conventional antidepressants, with LLLT providing a first round of benefit within about an hour of the initial irradiation and a second round of benefit within a few days of the initial irradiation.

Respecting highly acute events, Oron (supra, 2007) reports that in vitro red/NIR light increases ATP in neurons within 10 minutes of the application of red/NIR exposure, while Zhang reports that LLLT activates PKC in neurons within one hour of the irradiation (Zhang, supra, 2008). Thus, two mechanisms are acting favorably upon the PPD mother within an hour of LLLT treatment.

Respecting more subchronic events, Anders,2008 reports that red/NIR light increases BDNF in neurons within 3-7 days of the beginning of red/NIR light exposure. Zhang (2008)/Liang & Whelan (2006) report that red/NIR light increases bcl-2 in neurons within 6-28 hours respectively of the beginning of red/NIR light exposure. In contrast, Wada 2005 reports that only chronic use of lithium upregulates bcl-2 expression (Wada, J Pharmacol Sci. 2005 December;99(4):307-21.) and only chronic lithium correlates with clinical benefits (Wada 2005).

Further evidence of the quick acting nature of red light is found in studies examined the effect of red light upon neuronal sprouting. Wollman, Neurol. Res., 1998, 20: 470-2 reported that human cortical explants exposed to red/NIR light displayed significantly increased sprouting after only 6 days. Also, Rochkind, Lasers Surg. Med. 41, 277-281(2009) reported that 780 nm light irradiation of embryonic rat brain cultures induced rapid sprouting of nerve processes within 24 hours and a 3-fold increase in large perikaryae after 4 days. Rochkind reported that this “precocious appearance of large neurons is unlike the usual growth pattern in which neurons grow and become large only after several weeks in culture.” Rochkind found that irradiation at 50 mW for one minute induced the most significant change. Therefore, because of the red light irradiation-induced induction of early sprouting, it is believed that the NIR/red light device of the present invention may be able to provide a clinical benefit to the PPD patient within a few days of beginning treatment, thereby making it at least an attractive adjunct to conventional antidepressant therapy, and possibly an antidepressant supplanting technology.

The rapid nature of the response by LLLT in PPD may be beneficial to both the mother (in terms of relief from PPD) and the child (in term of gaining benefit from the increased positive interaction with the mother) during a critical period of life.

### DESCRIPTION OF THE FIGURES

FIG. 1 is a perspective view of the brain

FIG. 2 is a bottom view of the brain.

FIG. 3 is a bottom view of the brain indicating locations of activation in mothers' brains elicited by their infants' cues.

FIG. 4 is a bottom view of the brain, with a dashed line indicating the maternal zone (MZ).

FIG. 5 is a sggital view of the skull indicating the placement of the power source in the skin above the hairline and placement of the LED beneath the temporalis muscle.

FIG. 6a is a horizontal cross section of the anterior portion of the cranium, including the frontal margin.

FIG. 6b is a bottom view of the brain, with a dashed line indicating the maternal zone, and a triangle of circles indicating the frontal margin.

FIG. 6c is a bottom view of the brain, with a dashed line indicating the maternal zone, and an extended solid triangle indicating a 1 cm extension of the frontal margin.

FIG. 7A is a horizontal view of the cranium, with the device of the present invention implanted through the temporalis muscle.

FIG. 8a is a side view of the skull, showing a device of the present invention implanted through a BTE approach.

FIG. 8b is a bottom view of the brain, with the device of FIG. 8a.

FIG. 9a is a side view of the skull, showing a device of the present invention implanted through a coronal suture approach

FIG. 9b is a bottom view of the brain, with the device of FIG. 9a.

FIG. 10 is a horizontal view of the cranium, with the device of the present invention implanted through an orbitozygomatic approach and residing wholly in the frontal margin.

FIG. 11 is a device of the present invention having an LED and an Rf antenna.

FIG. 12 is a device of the present invention having an LED, battery and Rf receiving antenna.

FIG. 13 is a device of the present invention having an LED and a battery power source.

FIG. 14 is a schematic of representative electronics of the device of the present invention.

FIG. 15 is a cross-section of a device of the present invention.

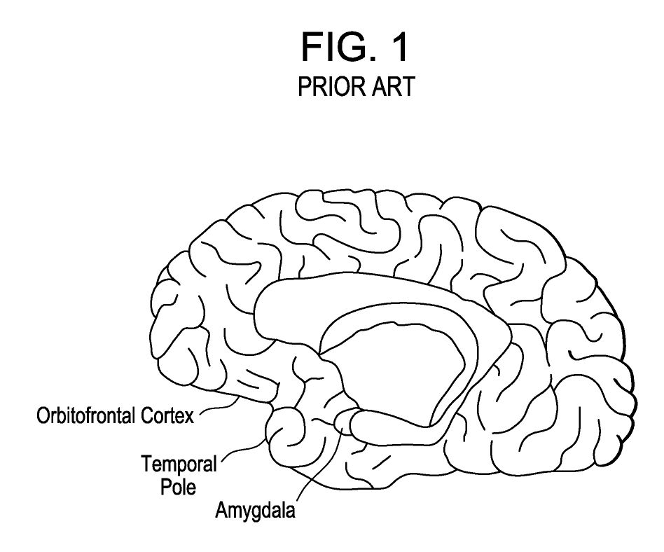

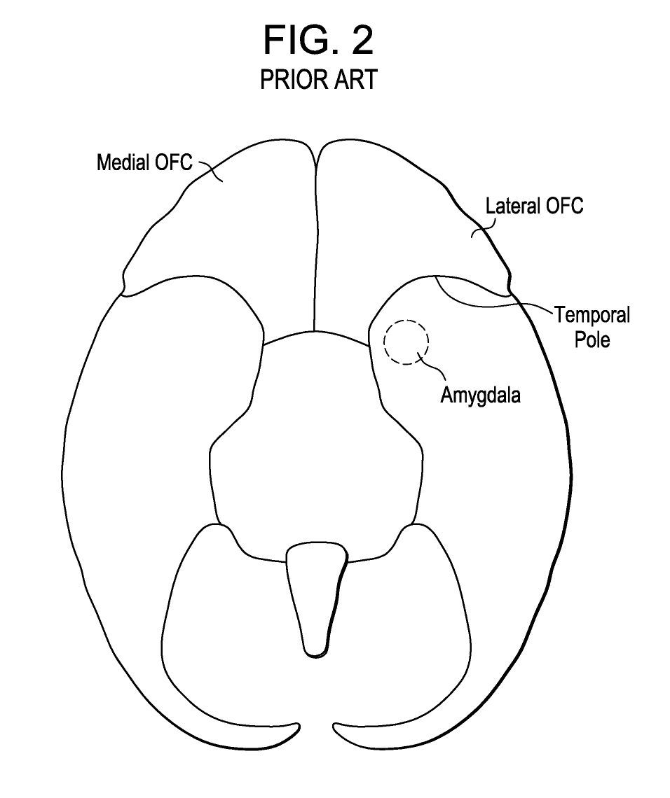

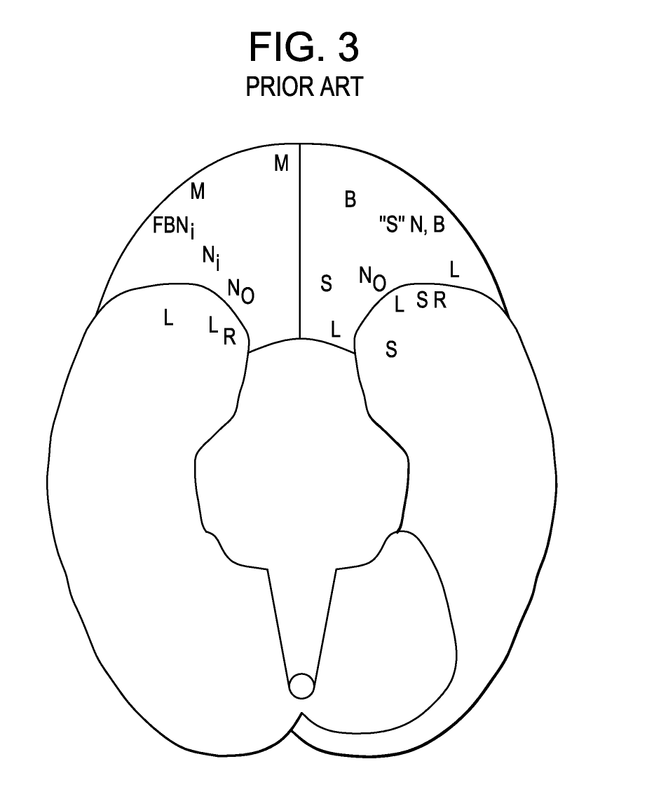

### DETAILED DESCRIPTION OF THE INVENTION

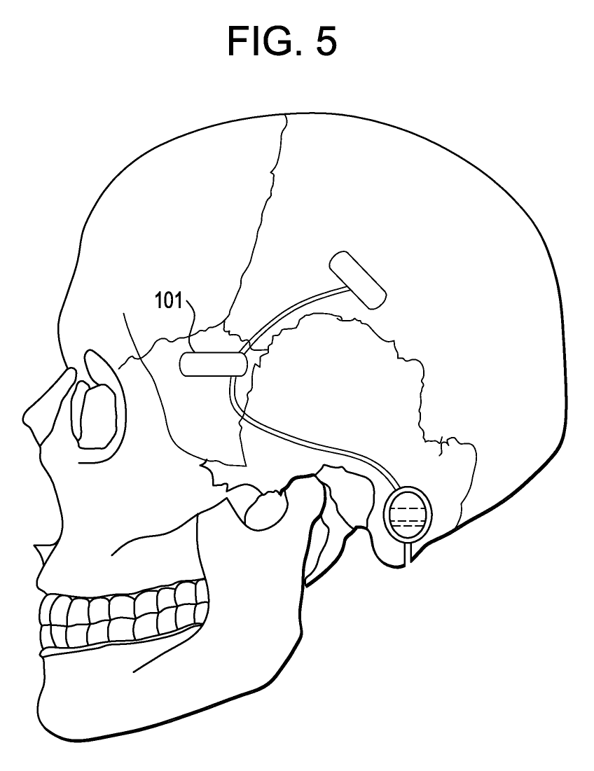

In some embodiments, the anterolateral portion of the MZ (i.e., the lateral OFC or cluster region) may be effectively irradiated by transcranial illumination. In one preferred embodiment targeting the cluster region, and now referring to FIG. 5, a red light device is placed under the temporalis muscle and upon a portion of the skull adjacent the cluster region. Placement of the device at this location is advantageous because only a small thickness of bone separates the device from the target cluster and so very little light attenuation occurs between emission of the light from the device and the target cluster region. This benefits the efficacy of the treatment while avoiding tissue overheating.

However, it is noted that the thin nature of the skull adjacent the temporalis muscle makes cleavage between these two masses very difficult. Therefore, additional device placement locations were sought.

In some embodiments, the posteromedial portion of the MZ (e.g., the amygdala) is irradiated by a fiber optic implanted within the lateral ventricle. However, intraventricular device placement requires a substantial invasion of the brain. Therefore, further additional device placement locations were sought.

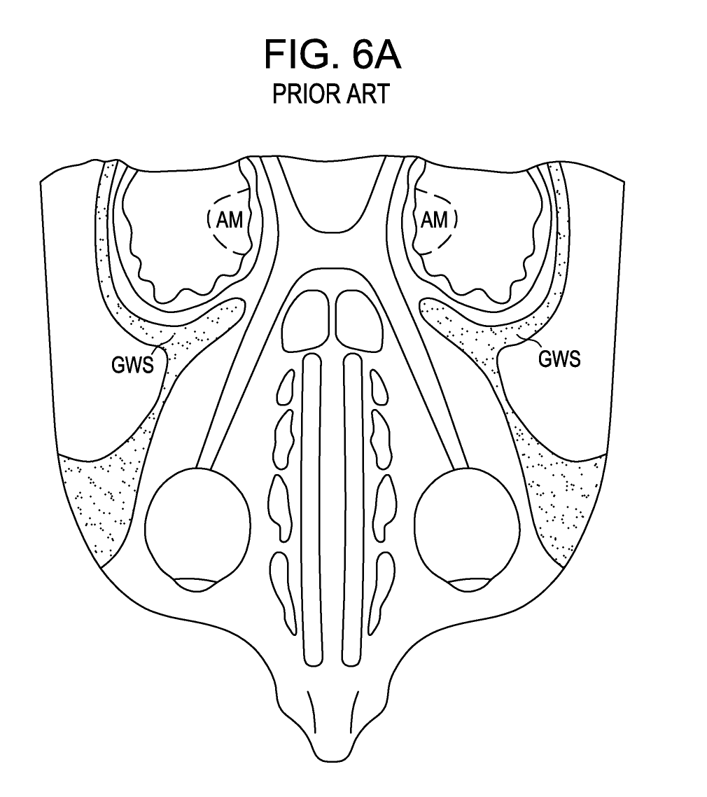

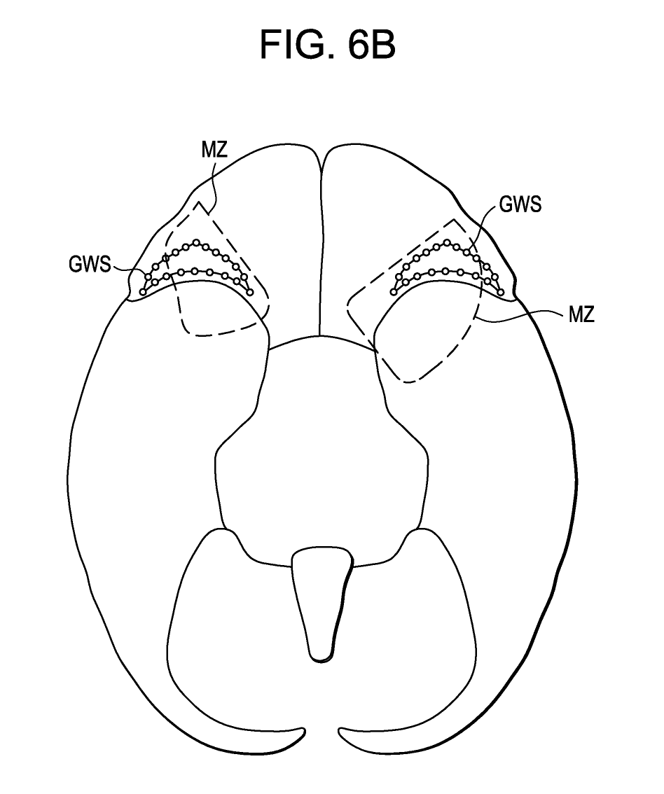

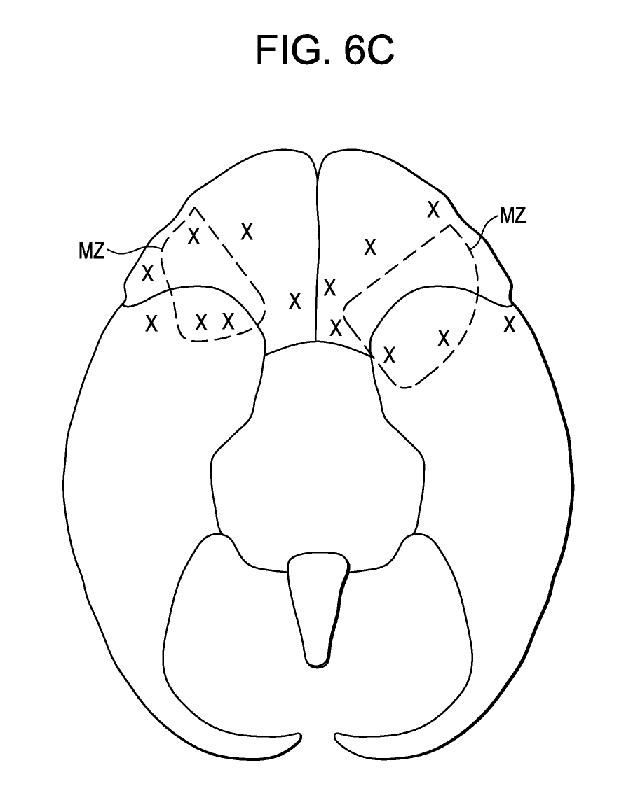

It has been further noticed that the central region of the MZ appears to sit directly on top of the bony roof of the greater wing of the sphenoid bone (GWS). See FIGS. 6a and 6b. The greater wing of the sphenoid bone (GWS) is essentially an egg-like bony structure comprised of a hard outer cortical shell and a softer, porous, cancellous bone interior. It sits beneath the posterolateral section of the orbitofrontal cortex, and sits directly in front of the temporal pole.

Because the GWS is located within about 1 cm of the lateral extremes of the MZ (see FIG. 6c), it appears that the GWS may be a very desirable location for placing an implant to irradiate the MZ.

Therefore, in some embodiments, the MZ is irradiated by placing a red/NIR light device within the GWS. Because the device of the present invention may be powered wirelessly via an external radiofrequency (Rf) power source, it is envisioned that the entire device may be so small so as to completely fit within the greater wing of the sphenoid bone.

In some embodiments, the procedure for placing the red light device adjacent the cluster region is carried out in a manner substantially similar to the conventional face lift procedure. That is, a small one inch incision is made in the skin in the upper temple above the hair line and the surgeon uses this site as access for lifting the temporalis muscle off the underlying skull. Next, a red light device is then inserted through the incision and placed upon the outer surface of the skull and beneath the temporalis muscle next to the cluster region. This procedure beneficially avoids harm to the temporalis muscle.

In some embodiments, the upper portion (frontal margin) of the greater wing of the sphenoid bone is accessed by a trans-temporal approach. In this approach, a minute incision is made in the temple of the patient, a few centimeters in front of the ear. Next, a cannula and in-dwelling stylet assembly is passed through the incision substantially transversely to pierce a small bore through the temporalis muscle to reach the lateral portion of the greater wing of the sphenoid bone. In some embodiments, the cannula has a diameter of less than 3 mm, preferably less than 2 mm.

Once the lateral portion of the greater wing of the sphenoid bone is reached, the pointed tip of the stylet is used to puncture the bone, thereby providing access to the interior of the greater wing.

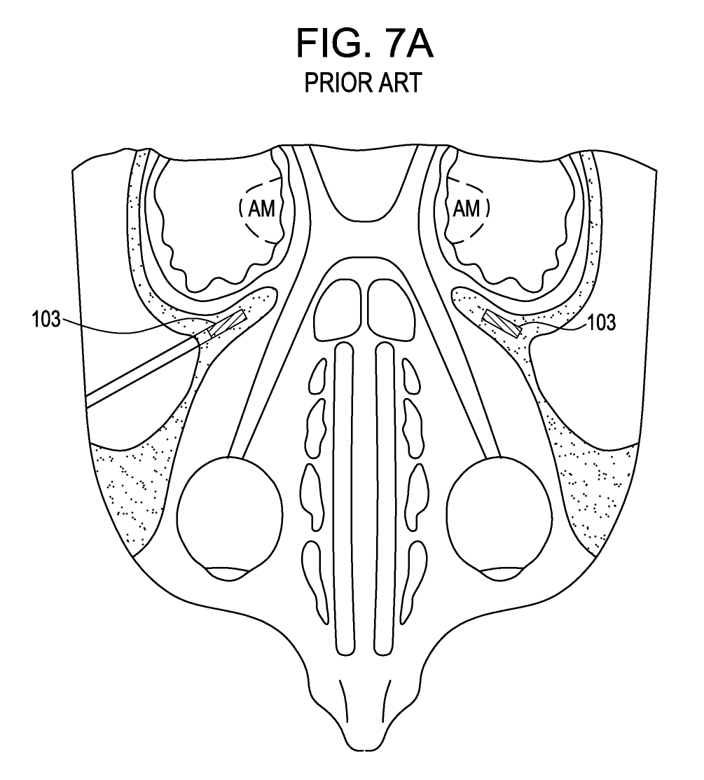

In some embodiments, the stylet is then removed from the cannula, the device 103 of the present invention is then inserted down the cannula, and is implanted into the upper portion of the greater wing. See FIG. 7A.

The major benefit of this approach is its simplicity and its relatively minimal invasiveness. In other embodiments accessing the GWS, the surgeon may also use the “face lift” metholofy discussed above in order to access the sphenoid bone and thereby avoid wounding the temporalis muscle.

It has been noted that the interior of the sphenoid bone may contain bone marrow and that bone marrow contains a high percentage of red blood cells with red light-absorbing hemoglobin. Thus, in some embodiments, the bone marrow present in the greater wing is first removed. The removal of this bone marrow removes the red light-absorbing hemoglobin from the system, thereby allowing red light to more easily pass from the device of the present invention to the cortical shell of the greater wing. Removal of the bone marrow may be conveniently accomplished by a lavage method. In some embodiments thereof, the lavaged volume is then filled with a substantially translucent cement. The translucency of this cement allows the red light to spread over a large surface area of the shell of the greater wing without substantial attenuation. In some embodiments, the cement is photopolymerizable and is activated by UV light.

In some embodiments, therefore, the device also has a UV LED present thereon that is adapted to inititate the above mentioned photopolymerization. Because the UV LED need only be activated once, in some embodiments, the device includes electronics that allow the UV LED to be turned off, or activated only once.

In some embodiments, any wound in the temporalis muscle is treated with LLLT. It has been reported that LLLT is beneficial for temporalis muscle-based pain.

Although GWS access is preferably accomplished through the temporalis muscle, other routes may also be used to gain access to the GWS.

For example, it has been further noted that the maxillary sinus (also called the antral chamber) is located essentially beneath the greater wing of the sphenoid bone, and that the maxillary sinus is routinely accessed by endoscopic procures. Therefore, in some embodiments, the device of the present invention may be placed into the GWS by intranasally or sublabially advancing the device into the maxillary sinus, and then piercing the appropriate portion of the posterior wall of the maxillary sinus to enter the greater wing of the sphenoid bone. In embodiments wherein the device is sublabially advanced, the procedures decribed in Ong, Neurosurgery 2008 March; 62(3 Suppl 1); 30-6, may be followed. However, in preferred embodiments, the intranasal route is followed, as it is a surgically less invasive procedure. When the intranasal route is followed, the device is first endoscopically advanced into the nasal cavity and is passed through the opening that connects the nasal cavity and the maxillary sinus. Because this opening typically has a diameter of about 2-4 cm, it is envisioned that widening the diameter of the opening may be necessary at times. However, this widening procedure is well known in the intranasal endoscopic art.

In some embodiments, the red light device residing in the antral chamber is held in place by an antral balloon. Antral balloons are very commonly used to stabilize and reduce fractures of the eye socket. See for example U.S. Pat. No. 3,800,788 (“White”), and DE 3736604. Arnaud, Orbit, 1989, 8(1) 57-63 reports that some antral balloons are commonly held in place for up to 12 days in order to allow the fracture to heal. Therefore, it is reasonable to expect that the balloon-fiber optic cable of the present invention will likewise be able to stay in place for up to 12 days as well. In this time period, it is believed that sufficient red light can be delivered to the neurons in the orbitofrontal cortex in order to promote sprouting and thereby increase the OFC gray matter in the targeted regions.

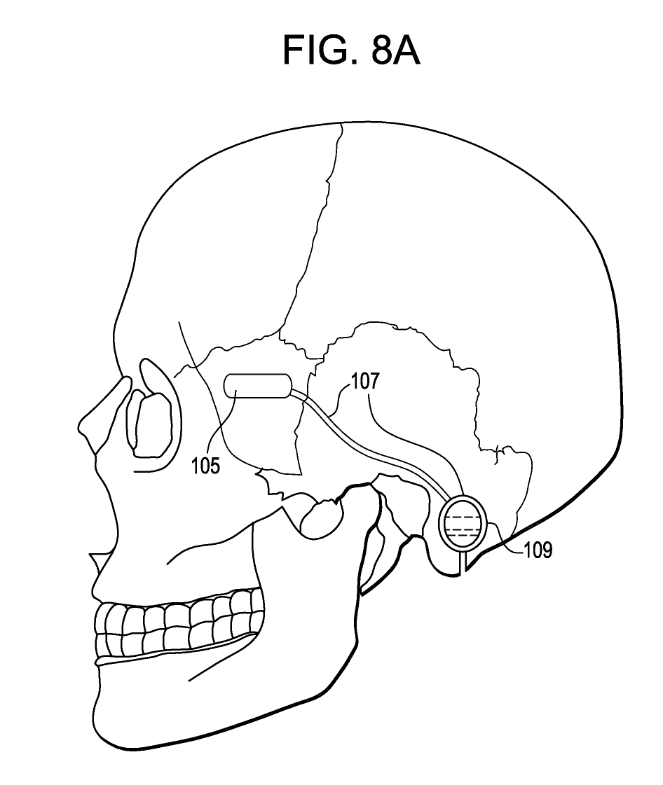

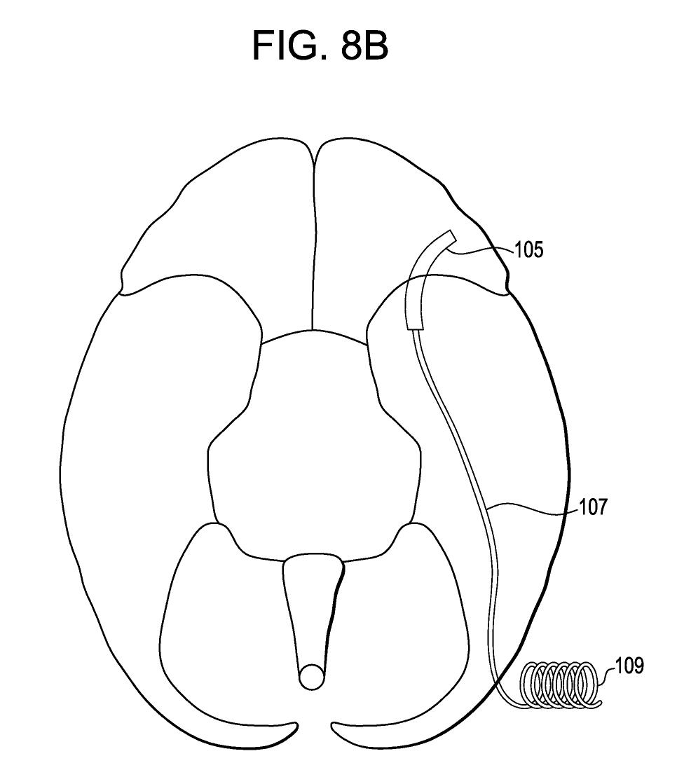

In other embodiments, access to the GWS is accomplished through an occipital burr hole. In these embodiments, and now referring to FIGS. 8a and 8b, a burr hole is first made in the occipital portion of the skull to expose the occipital portion of the brain. Preferably, the burr hole is made at a location just behind-the-ear (BTE). Procedures for making the BTE burr hole can be found in US Published patent Application No. 2005-0102006 (Whitehurst), the specification of which is incorporated by reference in its entirety. Next, a device having a distal strip of LEDs 105 is passed through the burr hole and is fluoroscopically advanced anteriorly over the temporal pole and to abut the OFC. Preferably, the strip of LEDs is of sufficient length such that the LEDs span the OFC portion of the MZ. More preferably, the strip of LEDs is of sufficient length such that the LEDs span the OFC and temporal pole portions of the MZ. Most preferably, the strip of LEDs is of sufficient length such that the LEDs span the OFC, temporal pole and the amygdala portions of the MZ. Once the strip of LEDs is properly located it is preferably sutured in place. In some embodiments, the device used in the occipital approach further has an intermediate lead wire 107 and a proximal Rf coil 109 adapted to be an antenna for reception of Rf energy from an external coil. The proximal Rf coil is preferably either placed within the burr hole or placed just beneath the skin surrounding the burr hole. In either case, the Rf coil of the device is preferably oriented parallel to the skin surface for maximum reception of externally delivered Rf energy. In some embodiments, the device placement procedure may be substantially similar to those epilepsy electrode placement procedures described in Steven, J. Neurosurg. 106: 1102-6, 2007, and US Patent Publication 2005-0102006 (Whitehurst), the specification of which is incorporated by reference in its entirety.

In other embodiments, a BTE incision is made and the LED is advanced outside the skull and beneath the temporalis muscle to site the LED just lateral to the cluster region.

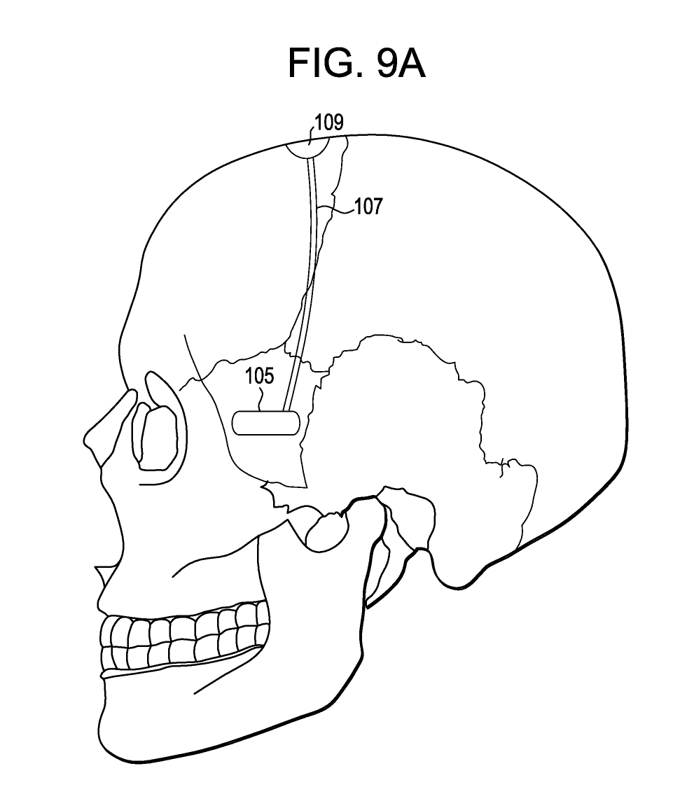

In some embodiments, and now referring to FIGS. 9a and 9b, the burr hole is placed just anterior to the coronal suture at the apex of the skull, and the device described immediately above is then fluoroscopically advanced across the temple region to the OFC. The coronal burr hole procedure has an advantage over the occipital or BTE procedure in that only a single burr hole need be made for placement of bilateral devices.

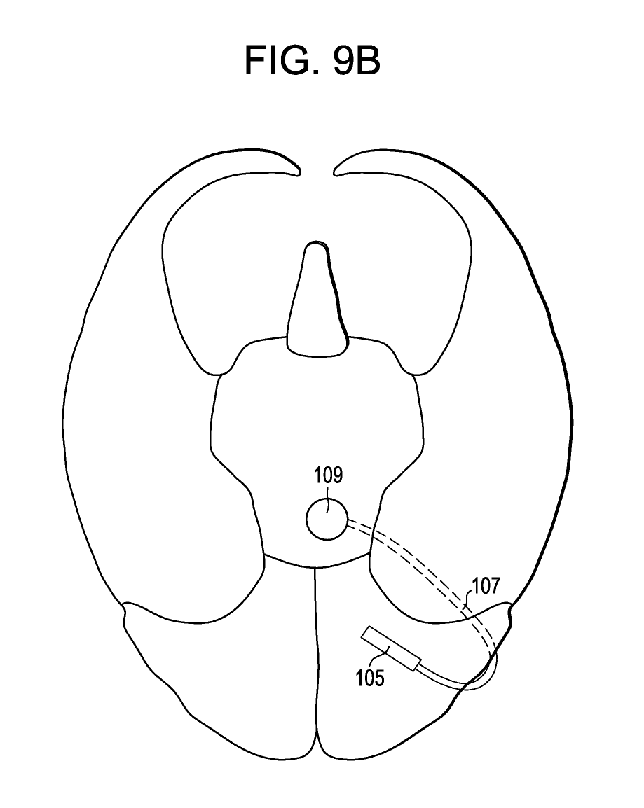

In some embodiments, and now referring to FIG. 10, a percutaneous orbitozygomatic procedure is carried out. In these embodiments, a small hole is made in the orbitozygomatic area, and a stylet-cannula assembly is fluoroscopically advanced through this small portion of bone so that it enters a cleavage place between the temporalis muscle and the lateral portion of the greater wing of the sphenoid bone. The stylet then pokes a hole in the upper, anterior portion of the greater wing. The LED 111 is then advanced down the cannula and into the greater wing of the sphenoid bone so that it sits directly adjacent the OFC. In some embodiments, the device used in the orbitozygomatic approach further has an intermediate lead wire 113 and a proximal Rf coil 115 adapted to be an antenna for reception of Rf energy from an external coil. The proximal Rf coil is preferably placed within the orbitozygomatic bone just beneath the skin. The Rf coil of the device is preferably oriented parallel to the skin surface for maximum reception of externally delivered Rf energy.

The orbitozygomatic approach is advantageous for two reasons: First, it follows a path that corresponds with a great portion of the locations identified by fMRI to be intimately associated with healthy and PPD mothers' response to infants cues. Therefore, this approach may allow for the most effective treatment of the PPD mother. Second, the placement of the Rf coil component of the device in the orbitozygomatic bone allows for the design of a large diameter Rf coil, thereby greatly increasing the amount of power available to the device. The greater power available, the more potential coverage of the MZ by red/NIR light from the device.

As noted above, both Minagawa-Kanai and Kringelbach reported distinctive activation of the anterior portion of the medial orbitofrontal cortex of mothers in response to photographs of their own and unfamiliar infants, respectively. It is noted that this distinctively activated area appears to lie directly behind the frontal sinus. Therefore, in some embodiments, the orbitofrontal cortex is irradiated by placing a NIR/red light source in the frontal sinus. Preferably, the frontal sinus is approached by accessing the nasal cavity.

In one preferred embodiment, the red light device is a probe comprising a red light wave guide, such as a fiber optic cable. The distal end of the fiber optic cable rests in the frontal sinus and is pointed towards the orbitofrontal cortex, while the proximal end of the fiber optic cable lies in the nasal cavity and is accessible from the nares. Therefore, the patient can apply red light to the proximal end of the fiber optic cable on a daily basis.

In some embodiments, the red light device comprises a balloon that expands when residing in the frontal sinus in order to keep the fiber optic cable in place. In some embodiments, the balloon and fiber optic cable are available from Acclarent.

In some embodiments, the device of the present invention has three basic components: a distal section comprising an LED, a proximal end comprising a wireless energy reception means (such as an Rf coil); and an intermediate portion providing an electrical connection therebetween (such as a wire or a pair of wires);

Preferably, the proximal portion of the device comprises a light emitting diode (LED) adapted to emit red/NIR light.

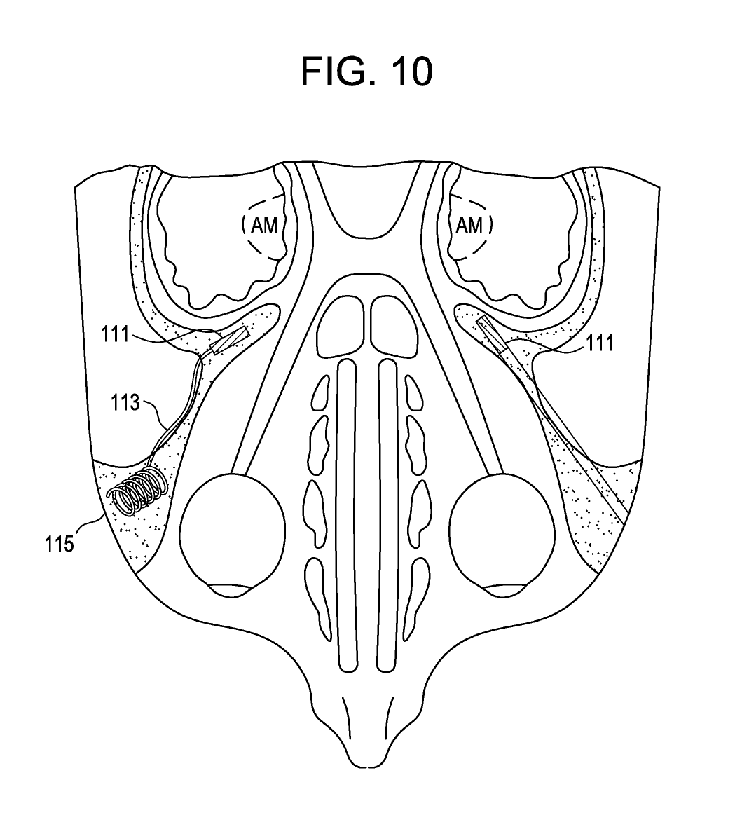

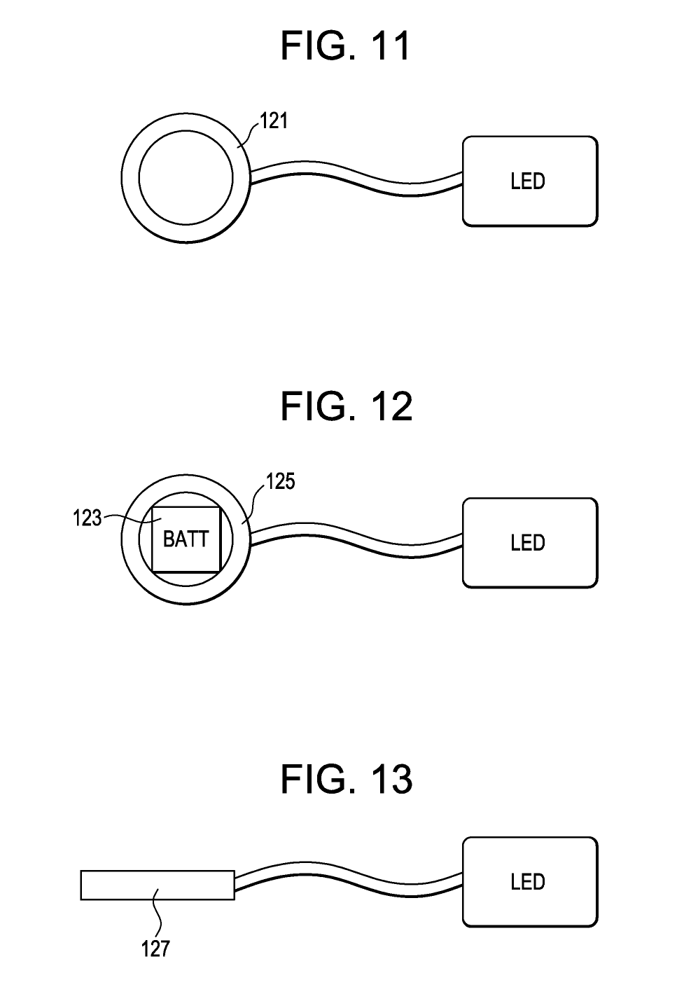

In some embodiments, and now referring to FIG. 11, the energy reception means comprises a receiving antenna 121 adapted to receive radiofrequency (Rf) energy and convert that Rf energy into a current.

In some embodiments, the energy reception means comprises a pair of leads, such as a cathode and an anode, that connect through the intermediate portion to the LED(s). In these embodiments, the pair of leads are placed close to the surface of the skin and spaced sufficiently far apart so that they can be coupled to a handheld bipolar electrode device . The bipolar electode device can then inject current into the device of the present invention in a safe manner. This embodiment has an advantage in that it has the potential to deliver high levels of energy to the LEDs.

Now referring to FIG. 12, in some embodiments, the red light device of the present invention is an implant having both a battery 123 and a Rf receiving antenna 125 or coil. The function of the battery in such an embodiment is to deliver energy to the red light LED. Because PPD typically lasts no more than about one year, and because four two minute treatments each day may be preferred, the battery should possesss a lifetime of at least about 50 hours. One preferred battery is the SR44, which has a diameter of about 10 mm, and a thickness of about 3 mm, and has a lifetime of about 66 hours.

The function of the Rf receiving antenna is to provide the power for turning the battery on. The patient (or clinician) can place the corresponding Rf transmitter on the skin overlying the implant and actuate the Rf transmitter, thereby transferring Rf energy to the device. This energy turns on the battery which in turns powers the NIR/red light LED. After about one to two minutes of therapy, a timer housed within the red light implant turns off the battery, thereby terminating therapy at an optimal treatment time. Preferably, each of the battery and the Rf receiving antenna are placed near the incision to facilitate repair and battery replacement, if needed.

In some embodiments, the patient wears a cap comprising a pair of bilaterally placed Rf transmitters powered by the same energy source. The energy source is controlled by a timer that regulates the frequency of power activations. In some embodiments, this cap-based timer prohibits activation of the bilateral Rf transmitters before the passage of about 6 hours. Therefore, the cap-based timer allows four well-spaced therapeutic irradiations per day.

In other embodiments, the battery 127 contains sufficient power so as to both power the LED for at least 50 hours and continuously run the timer, thereby allowing the Rf receiving coil to be eliminated from the device. See FIG. 13.

In some embodiments, the device of the present invention is based substantially upon the devices developed by Advanced Bionics Corporation. These devices, currently known as BION™ devices, are currently used as nerve stimulators.

The following documents describe various features and details associated with the manufacture, operation and use of BION™ implantable microstimulators, and are all incorporated herein by reference in their entireties:

a) U.S. Pat. No. 5,193,539, Implantable Microstimulator Mar. 16, 1993;

b) U.S. Pat. No. 5,193,540 Structure and Method of Manufacture of an Implantable Microstimulator Mar. 16, 1993;

c) U.S. Pat. No. 5,312,439 Implantable Device Having an Electrolytic Storage Electrode May 17, 1994;

d) U.S. Pat. No. 5,324,316 Implantable Microstimulator June 28, 1994;

e) U.S. Pat. No. 5,405,367, Structure and Method of Manufacture of an Implantable Microstimulator Apr. 11, 1995;

f) U.S. Pat. No. 6,051,017, Improved Implantable Micro-stimulator and Systems Employing Same. Apr. 18, 2000 and

g) U.S. Pat. No. 6,609,032

This invention teaches the electrical elements of an implantable, NIR/red light emitting LED useful in a wide variety of applications.

In some embodiments, the device of the invention is a very small NIR/red light emitting LED device and can be easily implanted, such as by injection through a hypodermic needle. It is operable to provide light of desired duration, desired fluence and desired intensity. The light pulses are delivered to the body through an LED provided near the outer surface of the device. Within the NIR/red light emitting LED device, an induction coil receives energy from outside the body and a capacitor is used to store electrical energy which is released to the device's LED under the control of electronic control circuitry means. The capacitor is controllably recharged to achieve “charge balancing”. Microelectronics are included within the device to provide the electronic control circuitry for controlling the various functions.

In some Rf embodiments, the device of this invention receives both energy and control information from a modulated, alternating magnetic field. A coil, acting as a secondary winding of a transformer, receives the alternating magnetic field energy which is rectified and stored on a capacitor. Regulation of the charge on the capacitor is provided. Electronic control means detects and decodes the modulating information to provide the desired control. Such control includes validating the received information, providing the light emitting signal, (its duration, fluence and intensity), and controlling the recharge of the capacitor for the stimulating light emission.

In some embodiments, there is provided a NIR/red light emitting LED device of a size and shape capable of implantation by expulsion through a hypodermic needle. In other embodiments, there is provided a NIR/red light emitting LED device which receives and utilizes an alternating magnetic field as a source of power. In other embodiments, there is provided an NIR/red light emitting LED device which receives a modulated, alternating magnetic field and detects the modulation to control a stimulating pulse. In others, there is provided an NIR/red light emitting LED device which effectively detects and demodulates the received, control signals.

Still another embodiment provides an NIR/red light emitting LED device in which the stimulating light features are controlled by a received alternating magnetic field. A final embodiment provides an NIR/red light emitting LED device in which energy is controllably stored in capacitor means, controllably discharged as a stimulating light pulse and which is controllably recharged.

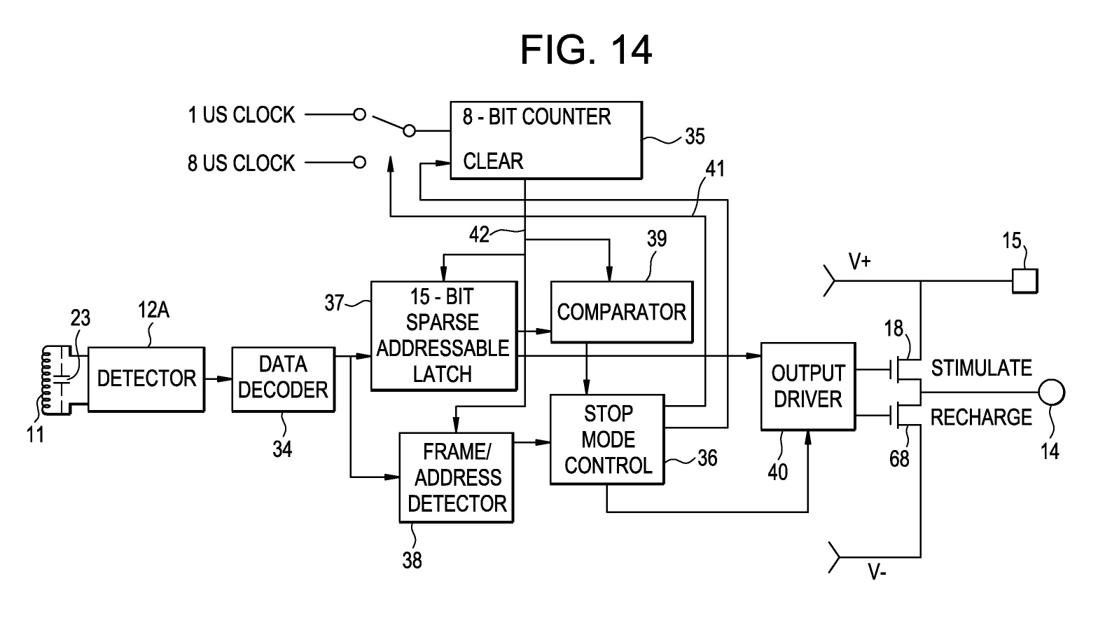

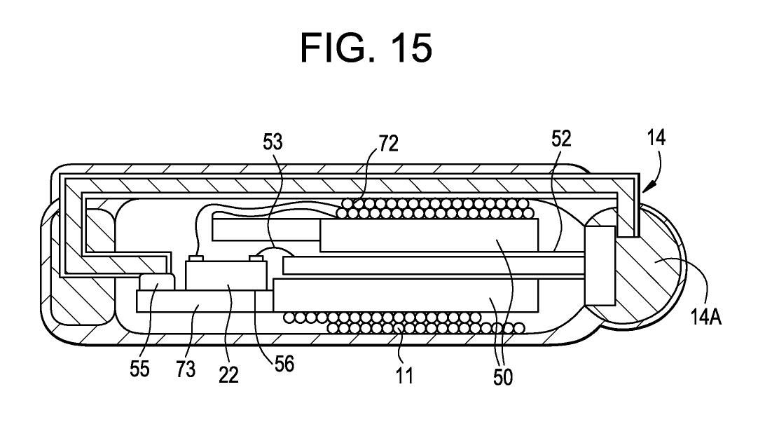

In some embodiments, the BION devices disclosed in U.S. Pat. No. 5,324,316 (Schulman) can be simply modified to produce an implant of the present invention. In one embodiment, the electrodes of the BION device are placed in electrical connection by a NIR/red light LED. One such modified device is shown in FIGS. 14 and 15 herein.

FIG. 14 is a block diagram illustrating one implementation of the circuitry of the electronic control means of the NIR/red light emitting LED device. Assuming a 2 mHz, modulated, alternating magnetic field is transmitted from outside the skin, coil 11 and capacitor 23 provide the signal at that frequency to data detector 12A, (part of a power supply and data detector). Assuming that the modulating information is contained in 36-bit frames, data detector 12A provides such 36-bit frame data to data decoder 34.

Data decoder 34 sends the data, to 15-bit sparse addressable latch 37 and the frame/address detector 38. Latch 37 is essentially a CMOS RAM storage device which stores only a portion of the received frame, in this instance, pulse duration and pulse amplitude. Location within a frame of data is signaled by the 8-bit counter 35 on line 42 to latch 37, frame/address detector 38 and comparator 39.

Latch 37 captures only those 15 bits relating to pulse width, pulse amplitude, range, recharge level and shape, (pulse tail or not), within a 36-bit frame. The latch 37 may be much like a CMOS static RAM with 6 transistors per storage cell.

Frame/address detector 38 looks at an incoming frame bit by bit and determines whether the information is addressed to this NIR/red light emitting LED device. It also checks the validity of the entire frame, which may be parity-encoded to insure accuracy. In the preferred embodiment, Manchester encoding of the bit transmission is also used.

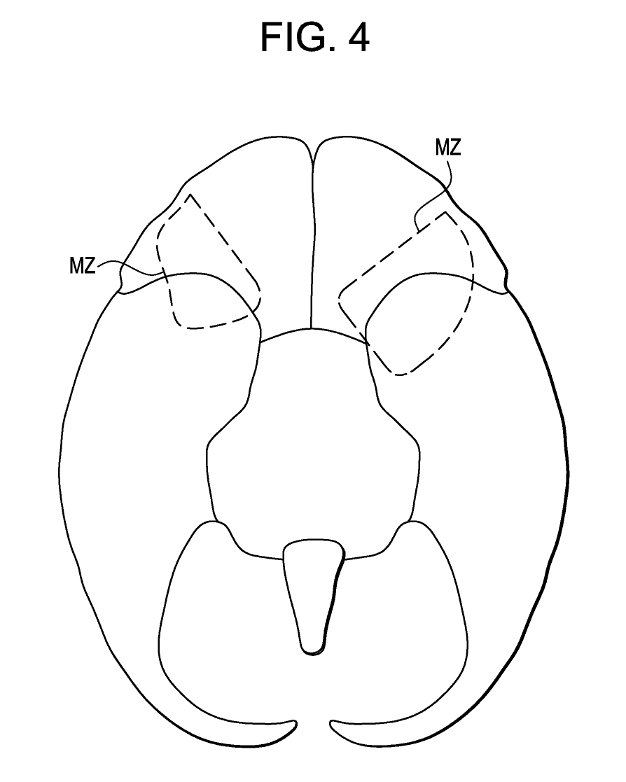

A frequency source (not shown) provides one or the other of two clock signals to 8-bit up counter 35, under the control of mode control 36. Such clock signals may be asynchronous with the 2 mHz frequency received from outside the skin. In a preferred embodiment, however, the clock signals are synchronous with the frequency of the received alternating magnetic field, by being derived from the received signal. This would be accomplished by clock signals which are obtained synchronously from the data decoder 34, from the alternating magnetic field frequency. As shown in FIG. 4, the asynchronous version, one clock signal is a 1 microsecond per count clock signal used in controlling the duration of the light emission and the other is an 8 microseconds per count clock signal used in developing and validating the data being received. Thus, the mode control 36 calls for one or the other of two modes, one mode, “generate light pulse” (the 1 microsecond per count mode) and the other mode, “search for valid frame”, (the 8 microsecond per count mode). Mode control 36, by controlling line 41, controls the input to 8-bit counter 35 and determines which count mode, 1 microsecond per count or 8 microseconds per count, is being received by the counter 35 from the frequency source.

During the 8 microsecond mode, if a valid 36-bit frame is not received by frame/address detector 38, or the address is not to this stimulator, the detector 38 resets itself, notifies mode control 36 which resets the 8-bit counter to zero and the search for a valid frame having a correct address commences again. If a valid 36-bit frame is received by detector 38, it notifies mode control 40 which switches to “generate pulse” mode, by clearing the 8-bit counter 35 and switching counter 35 to receive the 1 microsecond per count. The output driver 40 controls transistor 18 which is turned on to allow a stimulating light pulse for the requisite time as determined when comparator 39 determines that the count, from 8-bit counter 35, is equal to the stored count in latch 37. When such counts are equal, comparator 39 advises mode control 36 (that the pulse has been on the required time) and to stop. Mode control 36 then stops driver 40 which turns off transistor 18, so that it is non-conducting. While transistor 18 is turned on, tantalum lead 15 and iridium lead 14 are discharging a portion of the electrical charge between them, on the capacitor, thus providing a stimulating pulse to the LED.

Transistor 68 is controlled by output driver 40 to restore the full charge on tantalum lead 15 with respect to iridium lead 14, in preparation for the next stimulating light pulse. The recharge current is 100 microamps, in high recharge, and 10 microamps, in low recharge. The stimulating pulse amplitude may be, for example, 2 to 30 ma, in the high stimulation range, and 0.1 to 1.5 ma in the low stimulation range.

FIG. 15 is a cross-section side view of a NIR/red light emitting LED device. The first iridium lead 14 is located near the center of the device while the tantalum lead 15 is located at the left end of the device. They converge at opposite ends of the LED. The anodized layer 15A on tantalum lead 15 and the activated layer on iridium lead 14 provide a substantial capacitance between them when the device is implanted. To the right of the LED is a light transparent window 14A. The window can be configured to act as a dispersing lens.

In the manufacturing process, the exposed surface of lead 14 is activated by immersing it in a phosphate-buffered saline solution and touching its outside surface with a whisker probe, (fine iridium wire of 0.003″ D), and cycling for 20 to 30 minutes at 0.5 volts per second to a maximum of plus or minus 0.8 volts. The cyclic voltammetry builds up an electrically conductive layer of iridium hydrous oxide, layer 14A, (an activated layer), that is capable of being cycled reversibly between the +3 and +4 valence states, thereby transforming electron motion in the underlying metal into ion fluxes in the surrounding solution without inducing irreversible electrolysis of the water or metal. The interfacial impedance tends to be very low, also, reducing the necessary voltage between the electrodes 14 and 15 to be used in obtaining LED activation.

The coil 11 is shown wound around a ferrite core 50. Such core is cylindrical and is manufactured in two halves with a U-channel in each one. When such halves are placed together, a channel is thus formed between them. The iridium stem 52 passes through such channel. Integrated circuit chip 22 is carried on a ferrite shelf 73 and is connected to receive the output of the coil 11. Iridium lead 14 is connected through its stem 52 and wire 53 to receive the output of chip 22 and be controlled thereby. Tantalum lead 15 is connected through its stem 54 and through weld shim 55 to a metallized pad 56 on the shelf 73 of ferrite 50. Integrated circuit chip 22 sits on metallized pad 56 and is thus connected through its base, or substrate, to stem 54 of lead 15. Thus, leads 14 and 15 are both connected to receive the output of chip 22 and be controlled thereby. The integrated circuit chip 22, in a preferred embodiment, is double-poly, p-well (3 micron) CMOS in which the substrate is at the V+ supply rail to which the tantalum lead may be readily connected.

In another embodiment, weld shim 55 and metallized pad 56 are replaced by a small metallized pad on ferrite shelf 73 to which both the tantalum stem 54 and integrated circuit chip 22 are electrically connected, by flying bond wires or other means.

It is noted that such leads are hermetically sealed to housing 72. The preferred embodiment comprises a housing of N51A glass or other suitable biomedical grade capillary tubing having an inner diameter of about 1.25 mm's. It is available from or through glass fabrication houses such as Kimbel Glass, Corning Glass and others.

It is important to select a glass which is stable in body fluids, is transparent to NIR/red light, and which substantially matches the coefficient of thermal expansion of the tantalum and iridium because of the heating operations involved in fusing the electrodes to the glass housing.

In the case of the tantalum lead 15, the stem 54 is first fused to glass bead, or washer, 74, and then the glass bead 74 is fused to the housing 72. In the case of the iridium electrode 14, the ball portion provides a good sealing surface and is fused directly to the housing 72. In alternate embodiments, a simple platinum or titanium wire, or wire of other suitable metal, may pass through the end of such device and be fused to the housing 72 to provide a hermetic seal.

Therefore in some embodiments, there is provided an implantable NIR/red light emitting LED device substantially encapsulated within a hermetically-sealed housing which is inert to body fluids, said device comprising: a coil within said hermetically-sealed housing adapted to function as the secondary winding of a transformer, said coil adapted to be disposed in a modulated, alternating magnetic field, an NIR/red light emitting LED, at least two leads for providing electrical stimulation to the LED, electronic control means, capacitor means for storing power for said electrical stimulation, and for storing power for said electronic control means, means for controllably charging said capacitor means from said alternating magnetic field, said electronic control means controlling discharge of said capacitor means to provide electrical stimulation through said leads, and said electronic control means further providing control of at least one of the duration, the fluence and intensity of light pulses driven by said electrical stimulation in accordance with the modulation of said alternating magnetic field.

In other embodiments, there is provided an implantable NIR/red light emitting LED device comprising: a hermetically-sealed housing inert to body fluids wherein said LED is substantially encapsulated, said NIR/red light emitting LED device being of a size and shape adapted for implantation in selected areas of a body; a first inert metallic lead hermetically sealed to said housing at or near one end thereof and a second inert, metallic lead hermetically sealed to said housing at or near another end thereof; and a substantial portion of said leads being in electrical connection with the LED so as to provide stimulation pulses thereto; a coil having a multiplicity of turns of fine, electrically-conductive wire adapted to act as the secondary of a transformer and receive a modulated, alternating magnetic field; said alternating magnetic field providing power for said NIR/red light emitting LED device and said modulation providing control information for said NIR/red light emitting LED device; capacitor means for storing power received from said magnetic field; electronic circuitry; and means for electrically connecting said electronic circuitry in circuit with said coil, said capacitor means and said electrodes.

Further, they have such small dimensions (2-3 mm diameter, 15-25 mm length) that they can be injected through 10-13 gauge cannulae. These BION devices can be modified by the coupling of an LED to the BION devices' electrode to produce a device of the present invention.

In some embodiments, the device has a capacitor for storing energy. In these embodiments, the device can receive Rf energy from a wireless system at a relatively low rate (e.g., 1 mW), accumulate the energy in its capacitor, and then release the energy in the form of light at a relatively high rate (e.g., 10 mW) and in an amount suitable for providing LLLT.

In embodiments using a strip of LEDs, the diodes may be encapsulated in light transmissive silicone tubing, so that the silicone acts as a hermetic seal of the dioide and the red light may easily traverse thin silicone wall. In some embodiments, the silicon tubing may be impregnated with an antibacterial compound such as BACTISEAL™.In some embodiments, a shape memorized nitonal wire may be attached to outside of silicone tubing. When the wire is heated to body temperature, the nitonol wire deforms to a shape that increases its coverage of the MZ. In this way, the linear strip of LEDs can become a two-dimensional array of LEDs that covers a substantial portion of the MZ.

In some embodiments, the teachings and technology disclosed in U.S. patent application No. 12/041405, filed Mar. 3, 2008, entitled “Endoscopic Delivery of Red/NIR Light to the Substantia Nigra to Treat Parkinson's Disease” (COD5190), incorporated by reference in their entirety, are used.

Preferably, the red/NIR light of the present invention has a wavelength of between about 600 nm and about 1500 nm, more preferably between about 600 nm and about 1000 nm. In some embodiments, the wavelength of light is between 800 and 900 nm, more preferably between 825 nm and 835 nm. In this range, NIR/red light has not only a large penetration depth (thereby facilitating its transfer to the fiber optic and OFC), but Wong-Riley reports that cytochrome oxidase activity is significantly increased at 830 nm, and Mochizuki-Oda reported increased ATP production via a 830 mn laser.

In some embodiments, the wavelength of light is between 600 and 700 nm. In this range, Wong-Riley reports that cytochrome oxidase activity was significantly increased at 670 nm. Wollman reports neuroregenerative effects with a 632 nm He—Ne laser.

In some embodiments, the light source is situated to irradiate adjacent tissue with between about 0.01 J/cm² and 20 J/cm² energy. Without wishing to be tied to a theory, it is believed that light transmission in this energy range will be sufficient to increase the activity of the cytochrome c oxidase around and in the target tissue. In some embodiments, the light source is situated to irradiate adjacent tissue with between about 0.05 J/cm² and 20 J/cm² energy, more preferably between about 2 J/cm² and 10J/cm² energy.

The present inventor are aware of at least two reports of very favorable effects of red/NIR light irradiation of neuronal cells at fluences of less than 1 J/cm². As discussed above, Byrnes, Lasers Surg Med. 2005 August;37(2):161-71 found that a significant (P<0.05) increase in brain derived neurotrophic factor (BDNF) and glial derived neurotrophic factor (GDNF) in the 0.2 J/cm² group in comparison to the non-irradiated group. Oron, Photomed Laser Surg. 2007 June;25(3):180-2 reports that normal human neural progenitor (NHNP) cells were grown in tissue culture and were treated by Ga—As laser (808 nm, 50 mW/cm², 0.05 J/cm²). They found that the quantity of ATP in laser-treated cells 10 minutes after laser application was 7513+/±970 units, which was significantly higher (p<0.05) than the non-treated cells, which comprised 3808+/±539 ATP units. In sum, Oron found that the neuronal ATP level was essentially doubled by LLLT. In addition, Byrnes, Lasers Surgery Medicine, March 2005, 36(3) 171-85 reports that dosages as low as 0.001 stimulate cellular activity (such as DNA, RNA and protein production, proliferation and motility). Therefore, it is believed that fluences as low as about 0.01 J/cm² (and possibly even about 0.001 J/cm²) will be effective in providing therapy to the pertinent MZ neurons of the PPD patient.

In some embodiments, the light source is situated to produce about 10-90 milliwatt/cm², and preferably 7-25 milliwatt/cm².

In accordance with US Patent Publication 2004-0215293 (Eells), LLLT suitable for the neuronal therapy of the present invention preferably has a wavelength between 630-1000 nm and power intensity between 25-50 mW/cm² for a time of 1-3 minutes (equivalent to an energy density of 2-10 J/cm²). Eells teaches that prior studies have suggested that biostimulation occurs at energy densities between 0.5 and 20 J/cm². Wong-Riley. J. Biol. Chem. 2005 Feb. 11, 280(6), 4761-71 reports that fluences as high as 30 J/cm² appear to be effective in preventing cell death in neurons exposed to the mitochondrial poison KCN. In some embodiments, the preferable energy density of the present invention is between 0.1 and about 30 J/cm², more preferably between 0.5-20 J/cm², most preferably between 2-10 J/cm². In summary, a preferred form of the present invention uses red and near infrared (red/NIR) wavelengths of 630-1000, most preferably, 670-900 nm (bandwidth of 25-35 nm) with an energy density fluence of 0.5-20 J/cm², most preferably 2-10 J/cm², to produce photobiomodulation. This is accomplished by applying a target dose of 10-90 mW/cm², preferably 25-50 mW/cm² LED-generated light for the time required to produce that energy density.

It is further believed that red/NIR light irradiation of neurons will produce a significant upregulation in brain derived neurotrophic factor (BDNF) and glial derived neurotrophic factor (GDNF). Byrnes, Lasers Surg Med. 2005 August; 37(2):161-71 reports that olfactory ensheathing OECs were purified from adult rat olfactory bulbs and exposed to 810 nm light (150 mW; 0, 0.2, or 68 J/cm²). Byrnes found that a significant (P<0.05) increase in BDNF, GDNF and collagen expression in the 0.2 J/cm² group in comparison to the non-irradiated and high dose groups.

Of note, it has been reported that the neuroprotective effects of red/NIR light can be effected by a single irradiation on the order of minutes. Wong-Riley, J. Biol. Chem. 2004, e-pub Nov. 22, reports that irradiating neurons with 670 nm red light for only ten minutes results in neuroprotection. Similarly, Wong-Riley Neuroreport 12(14) 2001:3033-3037 reports that a mere 80 second dose of red light irradiation of neuron provided sustained levels of cytochrome oxidase activity in those neurons over a 24 hour period. Wong-Riley hypothesizes that this phenomenon occurs because “a cascade of events must have been initiated by the high initial absorption of light by the enzyme”.

Therefore, in some embodiments of the present invention, the therapeutic dose of red light is provided on approximately a daily basis, preferably no more than 3 times a day, more preferably no more than twice a day, more preferably twice a day.

In some embodiments, the red light irradiation is delivered in a continuous manner. In others, the red light irradiation is pulsed in order to reduce the heat associated with the irradiation. Without wishing to be tied to a theory, it is believed that pulsed light may be more effective in achieving the vibratory oscillation of the catalase and SOD molecules.

In some embodiments, red light is combined with polychrome visible or white light.

Thus, there may be a substantial benefit to providing a local radiation of the OFC with red/NIR laser light. The red light can be administered in a number of ways:

1) By implanting near the skull an implant having a red light LED, an antenna and a thin fiber optic terminating at the OFC, and telemetrically powering the LED via an external antenna to deliver red light through the fiber optic to the OFC.

2) By placing a fiber optic having a proximal light collector at the interior rim of the skull and running it to the OFC, and then irradiating the proximal end via an external red light source. NIR/Red light can penetrate tissue up to about one cm, so it might be able to cross the skull and be collected by the collector.

3) By implanting a NIR/red light LED in the skull, and powering the LED via an internal battery.

In each case, there is produced an effective amount of local red or near infrared irradiation around the OFC. This light would then increase local ATP production, and enhance SOD and catalase activity, thereby increasing the metabolism in and reducing the oxidative stress upon the OFC.

In use, the surgeon implants the device into the brain of the patient so that the device is adjacent to a portion of the OFC. The NIR/red light produced by the implant will then irradiate that portion of the OFC.

In order to protect the active elements of the device from cerebrospinal fluid (“CSF”), in some embodiments, the NIR/red light LED is encased in a casing. This casing both protects the LED components from the CSF, and also prevents the LED components from elicting an undesired immune reaction In some embodiments, the casing is made of a red/NIR light transparent material. The red/NIR light transparent material may be placed adjacent the LED component so that red/NIR light may be easily transmitted therethrough. In some embodiments, the transparent casing is selected from the group consisting of silica, alumina and sapphire. In some embodiments, the light transmissible material is selected from the group consisting of a ceramic and a polymer. Suitable NIR/red light-transmissible ceramics include alumina, silica, CaF, titania and single crystal-sapphire. Suitable light transmissible polymers are preferably selected from the group consisting of polypropylene and polyesters.

In some embodiments, it may be desirable to locate the light emitting portion of the implant at a location separate from the LED, and provide a light communication means between the two sites. The light communication means may include any of a fiber optic cable, a wave guide, a hollow tube, a liquid filled tube, and a light pipe.

In some embodiments, an implanted light communication means (or “optical wave guide”) is used to deliver photonic energy from the light source to a location within or adjacent the brain. The optical wave guide can be embodied as an optical fiber, or internally-reflective tube (or “light pipe”). At its distal end, it may have a diffusion/diffraction surface(s), an optical lens and/or a mirror system, etc. or a combination of these elements.

In some embodiments, the optical wave guide is a light pipe. In one embodiment, the light pipe is a truncated form of the Flexible Light Pipe FLP 5 Series, marketed by Bivar Inc., which is a flexible light pipe that is 12 inches long and 2 mm in diameter, and has an outer tubing of fluorinated polymer TFE.

In some embodiments, the optical wave guide is a coiled sheet or convoluted surface that guides optical energy (light) from a source to a final target (in this case, a tissue or anatomical region of the brain, the OFC). The benefit of a hollow optical wave guide is the decreased amount of light energy being absorbed by the material conduit. This benefit is mitigated by optical inefficiencies due to imperfect reflectance, but light attenuation by absorption will be greatly reduced in a hollow internally reflecting optical wave guide.

Silicone might also be used as the core and/or cladding of an optical fiber as long as the materials have different optical refractive indices. Those practiced in the art will appreciate how to manufacture silicone cores with silicone cladding.

Alternatively, a traditional optical material like glass or clear acrylic can be used as the optical wave guide core with silicone cladding that also serves as a biological boundary to impart overall device biocompatibility.

In some embodiments in which an optical wave guide is used, the distal end of the optical wave guide is a diffuser. Typically, the diffuser has a) a bulk phase comprising a light transmissive material and b) a dispersed phase comprising a light reflective material.

In some embodiments in which a fiber optic is used, the distal end of the fiber optic has a diffuser attached thereto.

In some embodiments in which a light pipe is used, the distal end of the light pipe has a diffuser or fiber optic cable attached thereto.

In some embodiments in which a fiber optic is used, the distal end of the fiber optic has a diffuser attached thereto.

In some embodiments, and now referring to FIG. 7, the prosthesis having an internal light source further contains an internal power source 300, such as a battery (which could be re-chargeable), which is controlled by an internal receiver and has sufficient energy stored therein to deliver electrical power to the light source in an amount sufficient to cause the desired light output.

When the implant is coupled with external energy, power can be transmitted into the internal device to re-charge the battery.

In some embodiments, the light source is provided on the implant and is adapted to be permanently implanted into the patient. The advantage of the internal light source is that there is no need for further transcutaneous invasion of the patient. Rather, the internally-disposed light source is activated by either a battery disposed on the implant, or by telemetry, or both. In some embodiments of the present invention using an internal light source, the light source is provided by a bioMEMs component.

Because use of the present invention may require its repeated activation by Rf energy, it would be helpful if the user could be guaranteed that the implant remained in the same place within the skull. Accordingly, in some embodiments, the device of the present invention comprises anchors, preferably projecting from the casing. Preferably, the anchors are placed on the proximal side of the device, adjacent the antenna. In this position, the anchor may be inserted into the bone of the skull S, thereby insuring its position.

In some embodiments, a red light source or red light collector and the proximal end of the fiber optic are placed in the chest. This allows the surgeon to conduct maintenance activity on an implanted light source without having to re-open the cranium. In addition, location within the chest also lessens the chances of surface erosion. In some embodiments, the distal end of the device can have a loop adapted to receive a suture so that the device may attach to the skull.

The formamen ovale is a bore in the skull base that opens onto the temporal lobe near the amygdala, and is commonly used as a minimally invasive conduit for percutaneous biopsy procedures involving the skull base. It is believed that the foramen ovale can be used as a desirable minimally invasive conduit for delivering a red/NIR light device that can suitable irradiate the amygdala and /or temporal lobe.

In some embodiments, the LLLT device is delivered endoscopically to the third ventricle, whereat it is able to therapeutically irradiate Brodmann's Area 25 (BA25).

It has been noticed that bipolar depression patients have also shown reduced gray matter in the cluster region (i.e., BA 47 or 47/12). See, for example, Nugent, Neuroimage, 30: 485-497, 2005; Rajkowska, Biol. Psychiatry, 45 1085-1098, 1999; and Lyoo, Biol. Psychiatry, 55, 648-651 (2004) Therefore, it is believed that the device of the present invention may further be helpful in providing therapy to bipolar patients as well, particularly in BA 47 or BA 47/12.

In addition to PPD, it is believed that red light irradiation of the “clsuer” region of the OFC may also be useful in treating major depression. Mak, Prog. Neuropsychopharmacol. Biol. Psychiatry, 2009 Oct. 1, 33(7) 1184-90 investigated gray matter volume (GMV) in female patients within major depressive disorder (MDD) and found a correlation in these depressed patients between reduced gray matter volume and difficulty in regulating negative emotion. Mak shows that the reduced gray matter volume resides in the cluster region of the OFC. Similarly, Lee, Prog. Neuropsychopharmacol. Biol. Psychiatry, 2008 Apr. 1, 32(3) 778-85 studied neural correlates of negative facial stimuli in patients with MDD and found that MDD patients showed decreased activation of the bilateral inferior OFC in response to these negative stimuli. Lee shows the decreased activation residing in the cluster region.

Therefore, two separate investigators have recently reported that patients with MDD that have difficulty in regulating negative emotion in response to negative stimuli have either reduced gray matter volume in the cluster region or reduced activation in the cluster region. Because it is believed that red light therapy will increase sporting in the gray matter regions irradiated, it is believed that red light therapy localized to the cluster region of the OFC will be therapeutic to MDD patients who have difficulty in regulating neragtive emotion.

We claim:

1. A method of treating depression in a patient, comprising the steps of:
a) inserting a device comprising a red/NIR light LED into a patient's skull,
b) activating the LED to irradiate at least a portion of an orbitofrontal cortex.

2. The method of claim 1 wherein the patient has post-partum depression.

3. The method of claim 1 wherein the device is inserted into an upper portion of a sphenoid bone in the patient and remains outside of a dura.

4. The method of claim 4 wherein the upper portion of the sphenoid bone is filled with a translucent synthetic material.

5. The method of claim 1 wherein the device is inserted between the patient's skull and a patient's brain.

6. The method of claim 1 wherein the insertion step is carried out through a temporalis muscle.

7. The method of claim 1 wherein the activation step includes transdermal transmission of Rf energy.

8. The method of claim 1 wherein the device includes a capacitor.

9. The method of claim 1 wherein the activation of the LED further irradiates an amygdala.

10. The method of claim 1 wherein the activation of the LED further irradiates a temporal pole.

11. The method of claim 1 wherein the orbitofrontal cortex is irradiated bilaterally.

12. The method of claim 1 wherein the orbitofrontal cortex is irradiated unilaterally.

13. The method of claim 1 wherein the insertion step include inserting the device through an occipital opening behind an ear of the patient..

14. The method of claim 1 wherein the insertion step includes inserting the device through a parietal opening anterior to a coronal suture.

15. The method of claim 1 wherein the insertion step includes intranasally inserting the device.

16. The method of claim 1 wherein the intranasal insertion step includes inserting the device into a maxillary sinus.

17. The method of claim 1 wherein the insertion step includes inserting the device through an orbitozygomatic approach.

18. The method of claim 1 wherein the irradiation results in an increase in metabolism in the orbitofrontal cortex.

19. The method of claim 1 wherein the irradiation results in an increase in cytochrome c oxidase in the orbitofrontal cortex.

20. The method of claim 1 wherein the irradiation results in an increase in BDNF in the orbitofrontal cortex.

21. The method of claim 1 wherein the irradiation results in an increase in bcl-2 in the orbitofrontal cortex.

22. A light-emitting implant for treating a brain disorder, the implant substantially encapsulated within a hermetically-sealed housing which is inert to body fluids, the implant comprising:
a) a coil adapted to function as a secondary winding of a transformer and to be disposed in a modulated, alternating magnetic field,
b) a red/NIR LED,
c) an electronic controller,
d) a capacitor for storing power for the LED and the electronic controller,
e) a charge controller for controllably charging the capacitor means from the alternating magnetic field,
wherein the electronic controller controls discharge of the capacitor to provide irradiation through the LED, and further provides control of at least one of the duration and the current in accordance with the modulation of the alternating magnetic field.

23. A light-emitting implant for treating a brain disorder, the implant comprising:
a) a planar array of red/NIR LEDs,
b) a power source in electrical connection with the LEDs,

24. A light-emitting implant for treating a brain disorder, the implant comprising:
a) a red/NIR LED,
b) a power source in electrical connection with the LED,
c) a non-linear wave guide connected to the LED.
